(19) United States
(12) Patent Application Publication
(10) Pub. No.: US 2025/0025668 A1
(43) Pub. Date: Jan. 23, 2025

(54) Catheter Securement Device

(71) Applicant: Rose Brunia Athime, Methuen, MA (US)

(72) Inventor: Rose Brunia Athime, Methuen, MA (US)

(21) Appl. No.: 18/354,861

(22) Filed: Jul. 19, 2023

(51) Int. Cl. A61M 25/02

(52) U.S. Cl. CPC A61M 25/02; A61M 2025/0266; A61M 2025/028; A61M 2205/18; A61M 2205/3306; A61M 2205/3334; A61M 2205/502

(57) ABSTRACT

A catheter securement device designed to enhance patient safety and streamline catheter management. The securement device is configured to attach around the exterior of a catheter tube and incorporates a digital clock for recording the catheter insertion date and tracking the time for catheter change. Additionally, the device features one or more sensors to monitor fluid flow within the catheter tube. This real-time fluid flow data enables early detection of blockages, reducing the risk of complications.

### BACKGROUND OF THE INVENTION

The present disclosure relates generally to a catheter securement device. More particularly, the present disclosure relates to a catheter securement device for automatically recording the time of catheter insertion and measuring the patency within the catheter.

Catheterization is a common medical procedure used to administer fluids, medication, or conduct diagnostic tests. To ensure proper patient care, it is crucial to maintain accurate and comprehensive records of catheter insertion, including the insertion date. However, existing practices often rely on facility policies for documenting catheter insertions, which can lead to inconsistencies and potential risks for patients.

Currently, the recording of catheter insertions largely depends on manual documentation by healthcare providers, which is susceptible to human error, misplaced records, or incomplete entries. This lack of standardized and reliable documentation creates challenges in tracking the duration of catheter usage, increasing the risk of catheter-related infections and related complications.

There are a number of other problems with current medical practice surrounding catheterization, to which there are a seemingly endless number of potential solutions. Therefore, what is needed is a catheter securement device for use on any type of catheter having all of the further described features and advantages.

### SUMMARY OF THE INVENTION

The subject matter of this application may involve, in some cases, interrelated products, alternative solutions to a particular problem, and/or a plurality of different uses of a single system or article.

In one aspect, a catheter securement device is disclosed. In this aspect, the securement device includes a main body configured to attach around the exterior of a catheter tube. The securement device also has a digital clock integrally connected to the main body, and the digital clock is operable to record a catheter insertion date and track a time for catheter change or removal. The securement device also includes an adhesive patch connected to the base of the main body.

In another aspect, a catheter securement device is also disclosed. In this aspect, the securement device includes a digital clock operable to record a catheter insertion date and track a time for catheter change or removal. This aspect also includes at least one sensor integrated into the catheter securement device, wherein the sensor is operable to monitor a fluid flow within the catheter tube.

### BRIEF DESCRIPTION OF DRAWINGS

FIG. 1 provides a perspective view of one embodiment of the catheter securement device attached to a catheter tube.

FIG. 2 provides a cross-sectional perspective view of one embodiment of the catheter securement device attached to a catheter tube.

FIG. 3a provides a perspective view of one embodiment of a digital display for a catheter securement device.

FIG. 3b provides a perspective view of one embodiment of a digital display for a catheter securement device.

FIG. 3c provides a perspective view of one embodiment of a digital display for a catheter securement device.

FIG. 4 provides a perspective view of another embodiment of a catheter securement device attached to a catheter tube on an appendage of a patient.

FIG. 5 provides a cross-sectional perspective view of yet another embodiment of a catheter securement device attached to a catheter tube.

### DETAILED DESCRIPTION OF THE INVENTION

The detailed description set forth below in connection with the appended drawings is intended as a description of presently preferred embodiments of the invention and does not represent the only forms in which the present disclosure may be constructed and/or utilized. The description sets forth the functions and/or the sequence of steps for constructing and operating the invention in connection with the illustrated embodiments.

Generally, the present disclosure concerns a catheter securement device that addresses critical issues associated with catheter usage and recordkeeping. Catheters are widely used in medical settings to deliver fluids, medications, or perform diagnostic procedures. However, failing to change a catheter in a timely manner can lead to serious risks for patients. One significant risk is the increased potential for catheter-related infections, which can be life-threatening and result in prolonged hospital stays and higher healthcare costs. Additionally, failure to change a catheter promptly may compromise its functionality and lead to complications, such as catheter obstruction.

Inconsistent recordation of catheter insertion dates further compounds the risks associated with catheter use. Many healthcare facilities rely on manual documentation practices, which are susceptible to errors and oversights. Misplaced or incomplete records make it difficult for healthcare providers to accurately track the duration of catheter usage, increasing the risk of infections and other complications. Without a standardized and reliable system for documenting catheter insertions, patient safety may be compromised, and the quality of care may suffer.

Catheter blockage is another critical concern. If a catheter becomes obstructed or partially clogged, it can impede the flow of fluids, leading to inadequate medication delivery or impaired drainage. Detecting blockages early is essential for timely intervention and preventing adverse patient outcomes. However, traditional catheter securement devices lack the capability to monitor fluid flow within the catheter, making it challenging to promptly identify blockages.

The catheter securement device described herein offers a solution to these problems. In most embodiments, the device is configured to attach around the exterior of a catheter tube, providing reliable securement. In some embodiments, the device incorporates a digital clock that efficiently records the catheter insertion date and tracks the time for catheter change or removal. In some embodiments, by automating the countdown timer through the use of an insulating pull tab, the device eliminates the need for manual tracking, reducing the risk of overlooking timely catheter replacements.

In one embodiment, the countdown timer may be initiated by removing a pull tab directly from the digital clock. The pull tab may be located on the clock's exterior, and when pulled, it may activate the timer circuit. This action may cause the digital clock to start tracking the time from the moment the pull tab is removed, ensuring that the countdown accurately reflects the catheter's dwell time or the total time since insertion.

In another embodiment, the countdown timer activation mechanism may be linked to the application of an adhesive patch connected to the securement device. In such an embodiment, the pull tab may be integrated into the base of the device and connected to contact paper covering an adhesive surface between the base and the paper. When healthcare providers adhere the adhesive patch to a patient's skin, the contact paper is removed, and the pull tab may be simultaneously pulled, thus initiating the timer. This integration ensures that the timer is activated only when the device is secured in place, minimizing the risk of pre-activation during storage or transportation.

In most embodiments, the catheter securement device includes at least one sensor capable of monitoring the fluid flow within the catheter tube. The sensor may be an optical or ultrasonic sensor operable to detect the flow rate and patency of the catheter. For instance, ultrasonic sensors can measure the time differential between bursts of ultrasound transmitted and received by the sensors, providing valuable data on the fluid flow velocity. Optical sensors, on the other hand, can analyze changes in light intensity caused by the fluid flow, allowing for real-time monitoring. Critically, both types of sensors are capable of monitoring fluid flow from outside the catheter tube. Accordingly, in most embodiments, the sensor and/or the sensor's components do not protrude into the interior of the catheter tube.

The integration of fluid flow monitoring capabilities into the securement device may ensure timely detection of catheter blockages. If a blockage occurs, the device may display an error message or an alert on the digital clock's display. Additionally, the device may sound an audible alarm through speakers, providing both visual and auditory notifications to healthcare providers and patients alike. This early warning system enables prompt intervention, preventing potential complications resulting from catheter blockages.

The monitoring of fluid flow may also provide another mechanism for activating the automated digital clock. For example, in yet another embodiment, the countdown timer may start automatically the first time a sensor detects fluid flow within the catheter tube. When the catheter is inserted and fluid begins to flow, the sensor may register this activity and trigger the timer circuit to commence. This approach may eliminate the need for manual initiation and may ensure that the timer starts precisely when the catheter becomes operational.

In one embodiment, the catheter securement device may be operable notify a healthcare facility when the catheter is due for change or if there is a blockage. This embodiment may include a non-transitory computer-readable medium stored within one of the digital clock or the main body of the securement device. The non-transitory computer-readable medium may contain instructions that allow and instruct a processor to execute the necessary steps for messaging as described herein.

Incorporated within the digital clock or the securement device, a transmitter component may facilitate communication with external systems, such as the healthcare facility. This transmitter may use electronic communication systems like a network, which could be a cellular communication network or the Internet. The network may serve as a system of interconnected electronic devices enabling data exchange, and the connections within the network may be wired or wireless.

When the countdown timer reaches the designated time for catheter change, or when a blockage is detected by the fluid flow sensor, the processor may receive this information. Based on predefined protocols and instructions stored in the non-transitory computer-readable medium, the processor may then trigger the transmitter to send notifications to the healthcare facility.

For example, if a catheter change is due, the processor may generate an alert message containing relevant patient and catheter information. This message may be transmitted through the network to the designated recipient at the healthcare facility, such as the nursing station or the responsible healthcare provider. The notification may serve as a timely reminder for catheter change, ensuring that the procedure is promptly carried out, reducing the risk of complications.

Similarly, in the event of a blockage, the processor may generate an alert message indicating a potential issue with the catheter's patency. This message may be sent through the transmitter and network to the healthcare facility, prompting healthcare providers to take immediate action to address the blockage and prevent further complications.

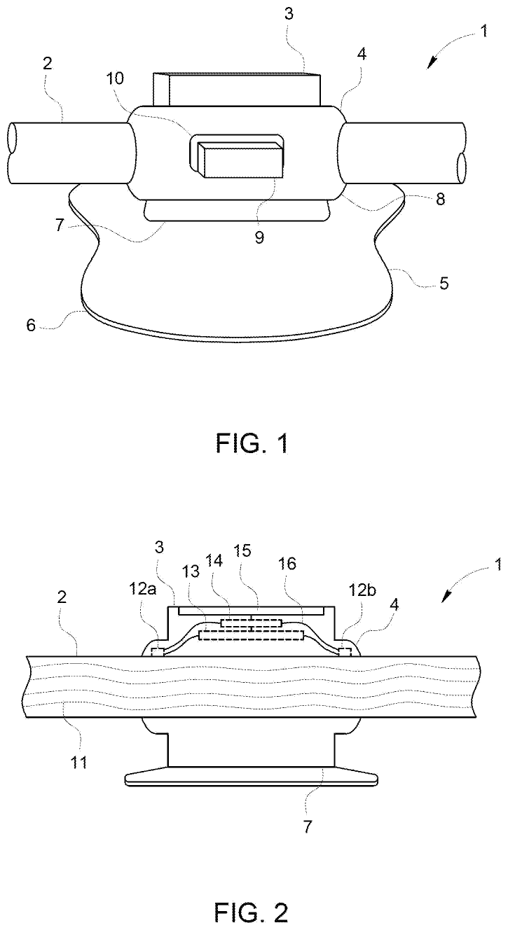

Turning now to the embodiment depicted in FIG. 1, the catheter securement device 1 is attached around the outside of a catheter tube 2. The securement device 1 includes several components, including, but not limited to a digital clock 3, a main body 4, and an adhesive patch 5.

The main body 4 serves as the central component of the device 1. It is constructed to provide stability, support, and comfort when attached to the catheter tube 2. The base 7 of the main body 4 is directly attached to the adhesive patch 5, which may facilitate secure attachment to a patient's skin. The adhesive surface of the patch is covered by contact paper 6 to maintain its integrity until the device 1 is ready for use.

To enhance the functionality of the device 1, a digital clock 3 is integrated into the main body 4. The clock 3 serves as a means to display the insertion date and time, providing a convenient and accurate method for documenting this critical information. This feature may eliminate the need for manual recording of catheter insertion date and time and may help ensure consistent and reliable results for patients.

The securing portion 8 of the main body 4 is designed to attach to a flange 9 connected to the base 7. The securing portion 8 includes an aperture 10, which allows for easy attachment and detachment of the device 1 from the catheter tube 2. This configuration may allow healthcare providers to securely fasten the device 1 in place while maintaining accessibility for catheter maintenance or replacement.

In FIG. 2, a cross-sectional perspective view of the catheter securement device 1 attached to a catheter tube 2 is presented. This embodiment of the device 1 incorporates additional features that may enable measurement of fluid flow 11 within the catheter.

Embedded within the main body 4 of the device, a pair of sensors 12a-12b are positioned to measure the fluid flow 11 within the catheter tube 2. The sensors 12a-12b are strategically placed and connected to electronic components housed within the digital clock 3. These electronic components include, but are not limited to, a battery 13 and a processor 14.

The battery 13 supplies electrical power to the sensors 12a-12b and the digital clock 3. The processor 14, connected to the sensors 12a-12b, receives and processes the data collected by the sensors. The information received from the sensors is displayed on the digital display 15 of the clock 3, offering real-time feedback on the fluid flow measurements.

To establish electrical connectivity between the various components, wires 16 directly link the sensors 12a-12b, battery 13, processor 14, and digital display 15. These wires 16 facilitate the transmission of signals and power, allowing for seamless operation of the device 1.

Still referring to FIG. 2, an exposed surface of the sensors 12a-12b comes into contact with the exterior of the catheter tube 2, facilitating accurate measurement of the fluid flow 11. In this embodiment, the sensors 12a-12b are ultrasonic sensors specifically designed for measuring fluid flow rates. The sensors 12a-12b are capable of both transmitting and receiving bursts of ultrasound waves. To measure the fluid flow rate, the sensors 12a-12b alternate between transmitting and receiving bursts of ultrasound between each other. Each sensor serves as both a transmitter and a receiver, allowing for bidirectional measurement of fluid flow.

When fluid is flowing through the catheter tube 2, a burst of ultrasound transmitted from the downstream sensor 12b will take a longer time to reach the upstream sensor 12a than a burst transmitted in the opposite direction. This time differential between the two bursts of ultrasound enables the calculation of the velocity of fluid flow 11 within the catheter tube 2.

Conversely, when fluid is not flowing, an ultrasound burst transmitted from either the upstream sensor 12a or the downstream sensor 12b will take the same amount of time to reach the receiving sensor. This equal time of flight between the two sensors may indicate that fluid flow has ceased or is no longer present in the tube 2.

By analyzing the time differentials and comparing them with a predetermined calibration, the processor 14 can accurately determine the fluid flow rate within the catheter 2. The calculated flow rate information can then be displayed on the digital display 15 of the digital clock 3, providing real-time feedback to the patient and/or healthcare providers.

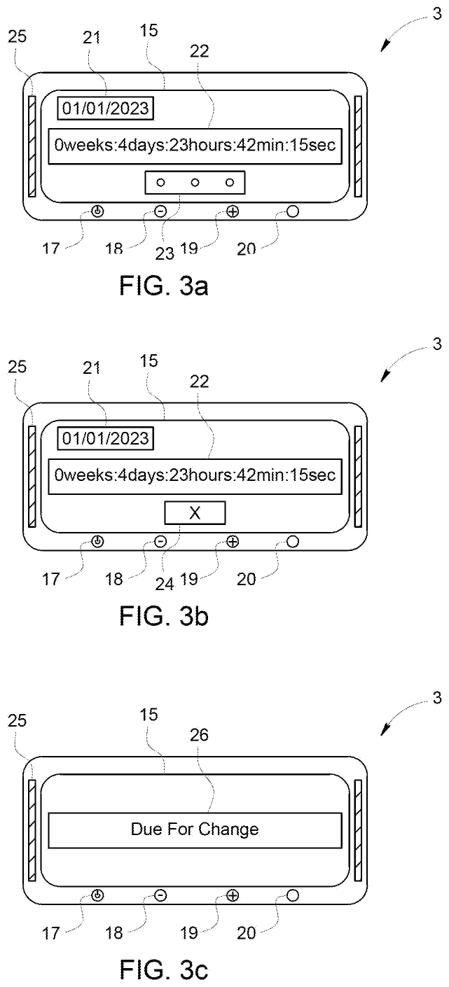

FIGS. 3a-3c showcase a perspective view of one embodiment of the digital display 15 for the digital clock 3. This display 15 incorporates various features to facilitate operation and customization as well as providing essential information and alerts, thus ensuring proper catheter maintenance and patient safety. In this embodiment, the power button 17 is featured proximate to the display 15 and serves as the main control for activating the digital clock 3. In a preferred embodiment, once the digital clock 3 is activated or turned on, it may not be operable to turn off or deactivate by only pressing the power button. This may ensure that the digital clock 3 will remain operational, despite inadvertent presses of the power button 17 by healthcare providers or the patient.

The clock 3 incorporates two adjustment buttons, 18 and 19, and a selection button 20, which may enable healthcare providers to configure specific items or settings shown on the digital display. The adjustment buttons, 18 and 19, may allow the user to navigate through various options and adjust parameters, such as time formats and other relevant settings based on facility policy or the patient's needs. The selection button 20 may allow healthcare providers to cycle through various options available for configuration and choose the desired display information.

The options available for configuration on the digital display 15 include, but are not limited to, the insertion date 21, the time remaining 22, and the patency of the catheter based on sensor information. As shown in FIG. 3a, when fluid is flowing unobstructed within the catheter, the clock 3 may show an indicator confirming the patency of the catheter on the digital display 15, such as, a plurality of steady green dots 23. As shown in FIG. 3b, in the event of a blockage within the catheter, the digital display 15 is operable to show an error message 24, for example, in the form of a blinking red X.

As the time remaining 22 on the digital clock 3 approaches the scheduled catheter change or removal time based on a predetermined time from the insertion date 21, the digital display 15 may provide a message 26 to alert the patient, as shown in FIG. 3c. Moreover, to further enhance patient safety and caregiver awareness, the digital clock 3 may be equipped with speakers 25 to emit alarm notifications when certain events occur. For example, if a blockage is detected, the device may produce an audible alert to notify nearby healthcare providers or the patient. Similarly, when the time for catheter change or removal is reached, an audible notification can be sounded to remind the patient and/or healthcare providers of the scheduled task.

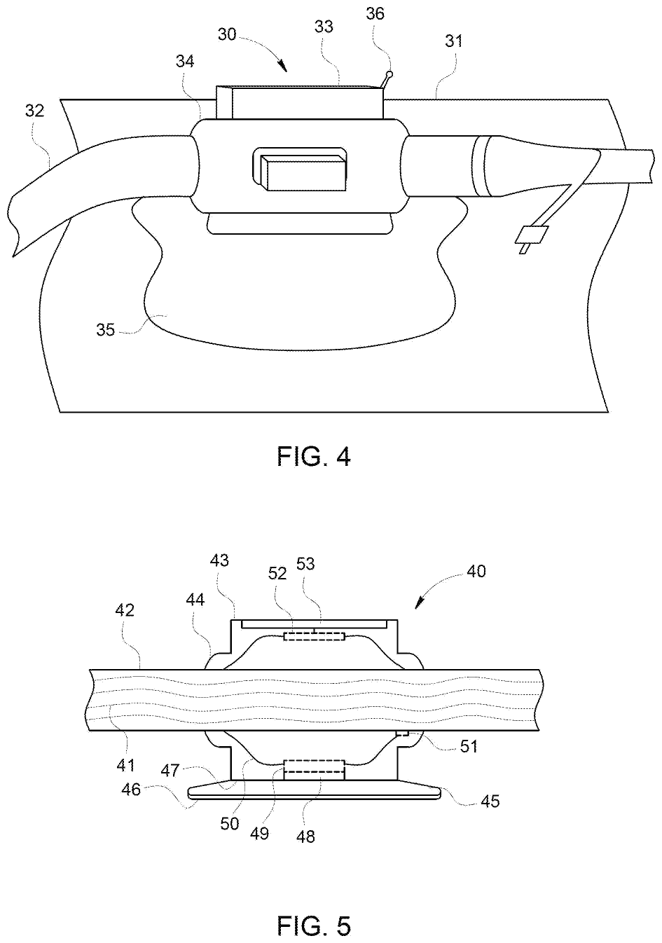

FIG. 4 provides a perspective view of another embodiment of a catheter securement device 30 attached to a catheter tube 32 on an appendage 31. The device 30 includes a digital clock 33 integrally connected to a main body 34, which is secured to an adhesive patch 35. The main body 34 secures the device 30 to the catheter 32, which in this embodiment is a foley catheter, and the adhesive patch 35 secures the device 30 to the patient's appendage 31, which in this embodiment is the patient's leg. However, it should be expressly understood that the securement device 30 is capable of attachment to any medical device having a tube that may require securement, patency checks, or deadline reminders, such as, suprapubic catheters, gastric tube feedings, and the like.

In this embodiment, the digital clock 33 features an automated countdown timer for tracking the time the catheter 32 is due for change or removal. This automated timer may eliminate the need for manual adjustments, making the catheter change process more efficient and reducing the risk of catheter-related complications. Typically, catheters only remain inserted for thirty (30) days, so the automated timer may be preset to notify the patient and/or healthcare providers upon the expiration of 30 days. However, the automated timer may be preconfigured to any time limit, based on facility policy and/or the patient's needs (e.g., four (4) weeks, six (6) weeks, eight (8) weeks, etc.).

To initiate the automated countdown timer, the digital clock 33 includes an insulating pull tab 36. When the device is prepared for use, the pull tab 36 can be removed from the digital clock 33 located on top of the main body 34. The act of removing the pull tab 36 may connect separate electric contacts within the digital clock 33, closing a circuit and activating the automated countdown timer. The countdown timer starts tracking the catheter's dwell time from the moment the pull tab 36 is removed. The digital clock 33 will provide a visual indication of the preset time remaining until the catheter is due for change, assisting healthcare providers in adhering to catheter maintenance schedules. This activation mechanism may ensure that the countdown timer is initiated precisely when the device is put into use, minimizing the risk of inaccurate tracking due to pre-activation during storage or transportation.

FIG. 5 shows a cross-sectional perspective view of another embodiment of a catheter securement device 40 attached to a catheter tube 42. The device includes a digital clock 43 integrally connected to a main body 44 and an adhesive patch 45 connected to the base 47 of the main body 44. In this embodiment, the catheter securement device 40 is designed to attach to a catheter tube 42 while incorporating additional features for fluid flow 41 measurement and automated countdown tracking.

The adhesive patch 45, connected to the base 47 of the main body 44, enables secure attachment of the device 40 to the patient's skin. The contact paper 46 covers the adhesive surface of the patch 45, ensuring its integrity until ready for use. The digital clock 43 includes an automated countdown timer for tracking the time the catheter is due for change. Similar to the previously mentioned mechanism, the automated timer is activated by removing an insulating pull tab 48 from the base 47 of the device 40.

In this embodiment, the insulating pull tab 48 is integrally connected to the contact paper 46. When healthcare providers adhere the adhesive patch 45 to the patient's skin, the contact paper 46 is removed, simultaneously exposing the adhesive and also removing the insulating pull tab 48 from the base 47. Upon removal of the pull tab 48, the battery 49 is activated, closing an electric circuit 50. This circuit 50 enables the activation of various electrical components connected to the battery 49.

The battery 49 is connected to at least the sensor 51 and the processor 52. The sensor 51 is responsible for measuring the fluid flow 41 within the catheter 42, while the processor 52 processes the data received from the sensor. The processor 52 is then operable to display this data on the digital display 53 along with countdown timer information. In addition to the sensor 51 and processor 52, other electrical components may be connected to the battery 49, depending on the specific functionalities and capabilities desired for the catheter securement device 40.

While several variations of the present disclosure have been illustrated by way of example in preferred or particular embodiments, it is apparent that further embodiments could be developed within the e spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is:

1. A catheter securement device comprising:
a main body configured to attached around an exterior of a catheter tube;
a digital clock integrally connected to the main body, the digital clock being operable to record a catheter insertion date and track a time for catheter change or removal; and
an adhesive patch connected to a base of the main body.

2. The catheter securement device of claim 1 further comprising at least one sensor integrated into the main body, the at least one sensor being operable to monitor a fluid flow within the catheter tube.

3. The catheter securement device of claim 2 wherein the at least one sensor is at least one of an optical sensor or an ultrasonic sensor.

4. The catheter securement device of claim 2, wherein the digital clock further comprises a display for indicating the fluid flow based on information received from the at least one sensor.

5. The catheter securement device of claim 2, wherein the digital clock further comprises an alarm operable to sound off when a blockage is detected by the at least one sensor.

6. The catheter securement device of claim 1, wherein the digital clock comprises an automated countdown timer, the automated countdown timer being activated by removing an insulating pull tab from the digital clock.

7. The catheter securement device of claim 6, wherein the adhesive patch comprises a contact paper.

8. The catheter securement device of claim 7, wherein the insulating pull tab is integrally connected to the contact paper.

9. The catheter securement device of claim 1 further comprising a battery connected to the digital clock.

10. The catheter securement device of claim 1, wherein the digital clock is operable to transmit alert messages through a transmitter.

11. A catheter securement device comprising:
a digital clock operable to record a catheter insertion date and track a time for catheter change or removal; and
at least one sensor integrated into the catheter securement device, the at least one sensor being operable to monitor a fluid flow within a catheter tube.

12. The catheter securement device of claim 11, wherein the at least one sensor is an optical sensor.

13. The catheter securement device of claim 11, wherein the at least one sensor is an ultrasonic sensor.

14. The catheter securement device of claim 11, wherein the digital clock further comprises a display for indicating the fluid flow based on information received from the at least one sensor.

15. The catheter securement device of claim 11, wherein the digital clock further comprises an alarm operable to sound off when a blockage is detected by the at least one sensor.

16. The catheter securement device of claim 11, wherein the digital clock comprises an automated countdown timer.

17. The catheter securement device of claim 16, wherein the automated countdown timer is activated when the at least one sensor detects the fluid flow within the catheter tube.

18. The catheter securement device of claim 11 further comprising a battery connected to at least one of the digital clock and the at least one sensor.

19. The catheter securement device of claim 11, wherein the digital clock is operable to transmit alert messages through a transmitter.

20. The catheter securement device of claim 11 further comprising a plurality of sensors, the plurality of sensors including at least one upstream sensor and at least one downstream sensor.
